(19) United States
(12) Patent Application Publication
(10) Pub. No.: US 2021/0046271 A1
(43) Pub. Date: Feb. 18, 2021

(54) NON-OBSTRUCTIVE NASAL CANNULA

(71) Applicant: Yeda Research and Development Co. Ltd., Rehovot (IL)

(72) Inventors: Noam SOBEL, Jaffa (IL); Ethan LIVNE, Rehovot (IL); Aharon WEISSBROD, Rehovot (IL); Sagit SHUSHAN, Rehovot (IL); Danielle Ricca HONIGSTEIN, Rehovot (IL)

(73) Assignee: Yeda Research and Development Co. Ltd., Rehovot (IL)

(21) Appl. No.: 17/083,493

(22) Filed: Oct. 29, 2020

Related U.S. Application Data
(63) Continuation of application No. PCT/IL2019/050470, filed on Apr. 28, 2019.
(60) Provisional application No. 62/674,648, filed on May 22, 2018.
(60) Provisional application No. 62/664,176, filed on Apr. 29, 2018.

(51) Int. Cl. A61M 16/06; A61M 16/00; A61M 16/08

(52) U.S. Cl. CPC A61M 16/0666; A61M 2230/42; A61M 16/0875; A61M 16/0003

(57) ABSTRACT

A nasal cannula includes a prong formed from an inner cylindrical wall surrounded by an outer cylindrical wall, a base connecting the inner cylindrical wall to the outer cylindrical wall and an air channel formed between the inner and outer cylindrical wall. The inner cylindrical wall defines a bore through which air may freely flow. The nasal cannula additional includes a connector fluidly connected to the air channel and configured to provide flow communication between the air channel and an external device connected to the connector.

### RELATED APPLICATIONS

This application is a US Continuation of PCT Patent Application No. PCT/IL2019/050470 having international filing date of Apr. 28, 2019 which claims the benefit of priority under 35 USC § 119(e) of U.S. Provisional Patent Application Nos. 62/664,176 filed on Apr. 29, 2018 and 62/674,648 filed on May 22, 2018. The contents of the above applications are all incorporated by reference as if fully set forth herein in their entirety.

### FIELD AND BACKGROUND OF THE INVENTION

The present invention, in some embodiments thereof, relates to a nasal cannula and, more particularly, but not exclusively, to a structure for a nasal cannula that does not obstruct the nasal passage.

A nasal cannula is a device used to supply gases directly to a patient's nose and is also used to measure gases from a patients nose. The gas may be supplemental oxygen or increased airflow and may be supplied to a patient or person in need of respiratory help. The nasal cannula is formed with a tube that is split into two prongs which are placed in the nostrils and through which the supply gas flows into the nostrils. The tube may be held in place with a hook around the patient's ears or with an elastic head band. A distal end of the tube may be connected to a gas supply source such as a portable oxygen generator or central supply source in a hospital via a wall connection. It is also known to use a nasal cannula for infants or neonates. Gas flow rate that may be achieved with a nasal cannula for infants or neonates may be limited due to the small dimensions of the nostrils. However, higher rates may be achieved with a known wider channel humidified nasal cannula. Another known drawback of nasal cannulae for infants or neonates is that due to the small dimensions of the nostrils, the prongs may plug the nostril and so that there is little or no free airflow passage between the nostril and the surrounding environment. This may cause significant side effects such as positive end-distending pressure to infants and significant alteration to their breathing strategy.

### SUMMARY OF THE INVENTION

According to aspects of some example embodiments, there is provided a nasal cannula that supplies gases to a patient's nose without obstructing the nasal passage and the course of natural breathing through the nose. The nasal cannula may be particularly suitable for neonates, infants and small children. The nasal cannula as described herein enables a patient, e.g. a neonate and an adult patient to freely exhale through their nose and also to supplement inhalation of gases delivered with the nasal cannula with air from the surrounding environment.

In some example embodiments, the nasal cannula is configured for sampling gases from the nasal cavity while the patient is breathing. Optionally, the nasal cannula is also configured for measuring changes in pressure in the nasal cavity during the breathing rhythm, and in this way measure breathing by providing a respiratory trace.

According to an aspect of some example embodiments, there is provided a nasal cannula comprising: a prong formed from an inner cylindrical wall surrounded by an outer cylindrical wall, a base connecting the inner cylindrical wall to the outer cylindrical wall, wherein the inner cylindrical wall, the outer cylindrical wall and base together define an open air channel between the inner and outer cylindrical wall and wherein the inner cylindrical wall defines a bore through which air freely flows; and a connector fluidly connected to the air channel and configured to provide flow communication between the air channel and an external device connected to the connector.

Optionally, the inner cylindrical wall has a first height and wherein the outer cylindrical wall has a second height and wherein the first height is less than the second height.

Optionally, the outer cylindrical wall has a truncated cone shape.

Optionally, the inner cylindrical wall has a first diameter and wherein the outer cylindrical wall has a second diameter at its distal end with respect to the base and wherein a ratio of the first diameter over the second diameter is 0.8-1.0.

Optionally, a width of the air channel tapers distal to the base.

Optionally, the connector is connected to the air channel via a port formed in the outer cylindrical wall.

Optionally, the base is configured to structurally support the prong and the connector and wherein the bore defined by the inner cylindrical wall extends through the base.

Optionally, the inner cylindrical wall is configured to be flexible and to collapse toward the outer cylindrical wall absent flow pressure in the air channel.

Optionally, the device is a source of compressed gas and wherein the air channel is configured to receive gas from the device via the connector and expel the gas through an opening between the inner cylindrical wall and the outer cylindrical wall.

Optionally, the prong is configured to provide flow communication from the opening between the inner cylindrical wall and the outer cylindrical wall to the device based on applying a vacuum through the connector.

Optionally, the nasal includes a second connector connected to a port in the inner wall, wherein the second connector is configured to provide flow communication between the bore and a sensor for sensing a parameter of breathing.

Optionally, the nasal cannula includes a pair of the prong, wherein the base connects the pair.

Optionally, the nasal cannula includes a pair of the connector, wherein each connector of the pair is fluidly connected to the air channel of one of the pair of the prong.

According to an aspect of some example embodiments, there is provided a method for providing respiratory assistance, the method comprising: providing a nasal cannula, the nasal cannula comprising: a prong formed from an inner cylindrical wall surrounded by an outer cylindrical wall, a base connecting the inner cylindrical wall to the outer cylindrical wall and an air channel formed between the inner and outer cylindrical wall, wherein the inner cylindrical wall defines a bore through which air may freely flow; and a first connector fluidly connected to the air channel and configured to provide flow communication between the air channel and an external device connected to the connector; a second connector connected to a port in the inner wall, wherein the second connector is configured to provide flow communication between the bore and a sensor for sensing a parameter of breathing; delivering air to the nasal cannula via the connector; and sensing the parameter of breathing based on connecting the second connector to the sensing device.

Optionally, the method includes extracting air from the second connectors and sensing the parameter of breathing based on the air extracted.

Optionally, the delivering and the extracting is performed concurrently.

Optionally, the delivering and the extracting is performed consecutively.

Optionally, the extracting is synchronized to occur at a defined period in a breathing cycle.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings (including an image). With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a perspective view of an example nasal cannula in accordance with some example embodiments of the present disclosure;

FIG. 2 is a top view of an example nasal cannula in accordance with some example embodiments of the present disclosure;

FIG. 3 is a cross-sectional view along a length of an example nasal cannula in accordance with some example embodiments of the present disclosure;

FIG. 4 is a same cross-sectional view of the example nasal cannula shown in FIG. 3 including indications of additional cross-sectional cuts;

FIG. 5 is section B-B of the example nasal cannula shown in FIG. 3;

FIG. 6 is section C-C of the example nasal cannula shown in FIG. 3;

FIGS. 7A, 7B, 7C and 7D is a perspective, side, front and top view respectively of another example nasal cannula in accordance with some example embodiments of the present disclosure;

FIG. 8 is a side view of the other example nasal cannula with arrows indicating example airflow directions in accordance with some example embodiments of the present disclosure;

FIG. 9 is a simplified block diagram of an example nasal cannula with associated sensors in accordance with some example embodiments;

FIG. 10 is an image of an infant fitted with an example nasal cannula and a graph of example measured flow both in accordance with some example embodiments of the present disclosure; and

FIGS. 11A, 11B and 11C are images of a child fitted with the example nasal cannula.

### DESCRIPTION OF SPECIFIC EMBODIMENTS OF THE INVENTION

The present invention, in some embodiments thereof, relates to a nasal cannula and, more particularly, but not exclusively, to a structure for a nasal cannula that does not obstruct the nasal passage.

A known drawback of nasal cannulae is that nasal cannulae tend to block a significant portion of the nasal passage due to the prongs that are placed in the nostrils. For neonates and children the blockage is even more significant and may reach 80% of the nasal passage. The blockage may make exhaling through the nose cumbersome and difficult. Adults may alter their natural breathing pattern and instead exhale orally. Neonates being obligate nose breathers cannot compensate by exhaling orally and therefore may not succeed in exhaling properly. Disruption of proper exhalation in the neonates may lead to end-distending pressure buildup that in turn may lead to gastrointestinal perforations. Another concern associated with pediatrics and especially neonates, regarding usage of a nasal cannula, is a risk that if the supply through the nasal cannula is terminated unexpectedly, the blockage due to the prongs may significantly impair the ability of the neonate or baby to take in air from the surrounding environment.

According to an aspect of some example embodiments, there is provided a nasal cannula that does not significantly obstruct the nasal passage. According to some example embodiments, the non-obstructive nasal cannula may be used for adults, children, infants as well as neonates and may support maintaining a natural breathing pattern. According to some example embodiments, the non-obstructive nasal cannula includes a pair of ring shape prongs, each of which define an annular air channel through which air may be delivered and a central bore through which a patient may have free flow communication with the surrounding environment. In some example embodiments, each prong is formed from a pair of substantially concentric cylindrical walls and a base that together define an air channel between the concentric cylindrical walls through which gasses may be actively delivered to the patient and/or sampled from the patient. The pair of substantially concentric cylindrical walls includes an inner wall that defines the central bore and an outer wall surrounding the inner wall. According to some example embodiments, both the inner wall and the outer wall extend from a ring shaped base. Flow communication with a gas source may be via a port formed in outer wall or on the ring shaped base and a tube connected to the port.

In some example embodiments, the outer wall is shaped as a truncated cone and the inner wall is defined to have a constant diameter with a height less than the outer wall. Optionally, a channel formed between the inner and outer wall is angled toward the inner wall. In some examples, a diameter of the outer wall distal from the base is substantially equal to a diameter of the inner wall. In this manner there is substantially no obstruction of the nasal passage due to the outer wall and annular air channel.

In some example embodiments, the inner wall may be formed from flexible material and may be configured to collapse toward the outer wall while no active flow is being delivered. Optionally, based on flow pressure during delivery of gas from gas source the inner wall may open the channel between the walls, e.g. move away from outer wall. By collapsing the inner wall while no active flow is being initiated, a diameter of the bore may be increased to further accommodate free flow communication with the surrounding environment.

The nasal cannula may be used as a respiratory assisting device, may be used as a sensing device for monitoring breathing parameters and may be used as both a respiratory assisting device and a sensing device. In some example embodiments, the nasal cannula includes a dedicated air channel through which sensing may be performed concurrently with respiratory assistance. Optionally the dedicated air channel may be fluidly connected to the central bore formed in the cannula and may sense air flow through the central bore during a breathing cycle. Alternately or additionally, sensing may be performed through an air channel through which gas may be delivered to the nasal cavity, e.g. the air channel that is fluidly connected to the ring shaped cavity formed in the nasal cannula.

Optionally, sensing with the nasal cannula may include for example extracting gases from the nasal cavity by applying a vacuum at an end of the tubing distal from the cannula and analyzing parameters of the extracted gases. Sensing may also include for example monitoring pressure changes. Pressure measurements may be monitored by connecting a sensor at the end of the tubing distal from the cannula.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

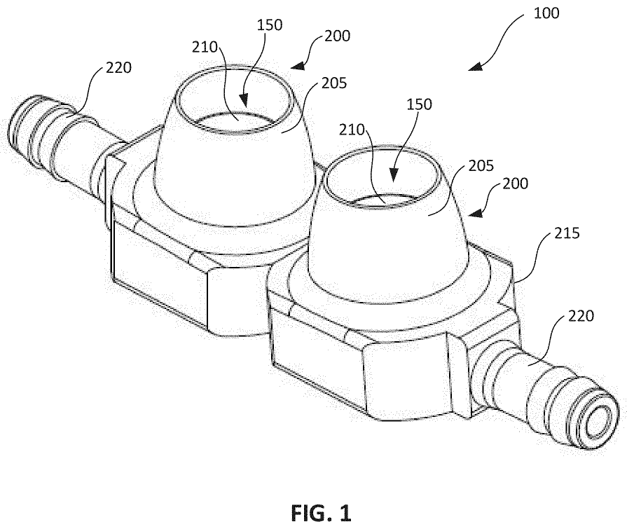

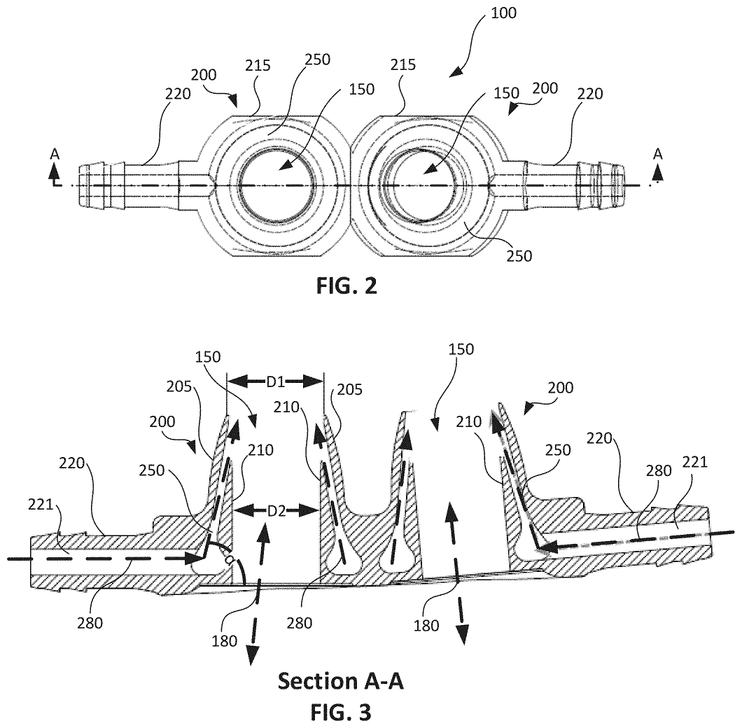

Reference is now made to FIG. 1, FIG. 2 and FIG. 3 showing a perspective, top and cross-sectional views of an example nasal cannula respectively, in accordance with some example embodiments of the present disclosure. A nasal cannula 100 may include a dedicated prong 200 for each of a right and left nostril, a dedicated connector 220 for each of a right and left nostril through which air supply is delivered to prong 200 and optionally a base 215 configured to provide structural support for prongs 200 and connectors 220.

According to some example embodiments, each prong 200 includes a ring shaped cavity 250 or channel formed between an outer wall 205 and an inner wall 210 of prong 200 as well as a central bore 150 through which air may freely flow (FIG. 2 and FIG. 3). Ring shaped cavity 250 is an air channel through which air or gas may be actively delivered to nasal cavity or optionally extracted for sensing. Connector 220 may be integrated or connected to outer wall 205 and may provide flow communication between ring shaped cavity 250 and an external device. Typically, flexible tubing may be connected to connector 220 at a first end and to a device, e.g. a source of compressed air or a sensing device at an opposite end. In some example embodiments, outer wall 205 may be shaped as a truncated cone or may generally taper as it extends out from base 215. The truncated cone shape may accommodate fitting nasal cannula 100 into different size nostrils and may also reduce potentially obstruction of natural airflow as described in more detail herein below. Optionally, nasal cannula 100 may be sized to slightly expand a nostril when worn. An extent of the expansion may be based on a depth at which prongs 200 are pushed into the nostrils.

In some example embodiments, nasal cannula 100 may be formed from a polymer material. Optionally, nasal cannula 100 may be formed based on injection molding.

Referring now to FIG. 3, a diameter D2 of bore 150 may be sized to provide a relatively wide opening through which a patient may naturally breath (inhale and exhale) with airflow 180 simultaneously with receiving supplemental oxygen or increased airflow 280 with nasal cannula 100. Optionally, tapering of outer wall 205 provides a diameter D1 at the exit that is equal to diameter D2. In this manner, obstruction of natural air exchange (airflow 180) may be substantially minimized. In some example embodiments, a ratio of D2 and D1 may be defined to be 0.7-1.0.

In some example embodiments, inner wall 210 is defined to be lower in height than a height of outer wall 205. Cavity 250 between inner wall 210 and outer wall 205 provides flow communication between a patient and a pressurized gas source connected to connectors 220. Optionally, outer wall 205 is angled toward inner wall 210. Optionally the angle of inclination a may be 60°-85°. By angling outer wall 205 and defining inner wall 210 to be lower in height than outer wall 205, a diameter D2 of bore 150 may substantially reach diameter D1. In this manner, controlled airflow 280 may be received in the nasal cavity (or extracted from the nasal cavity) without obstructing free airflow 180 via the bore 150.

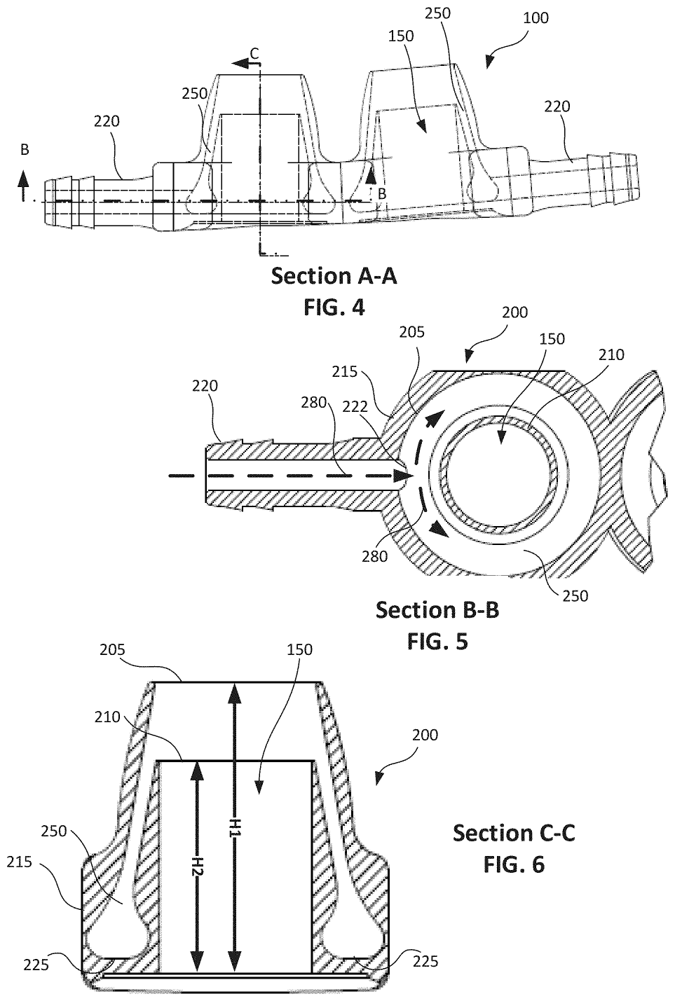

Reference is now made to FIG. 4, FIG. 5 and FIG. 6 showing additional cross-sectional views of the example nasal cannula in accordance with some example embodiments of the present disclosure. According to some example embodiments, controlled airflow 280 enters prong 200 from a lateral direction and spreads through a ring shaped cavity 250 around bore 150. Optionally, cavity 250 is a channel formed in base 215 and that extends upwards between inner wall 210 and outer wall 205. Optionally, cavity 250 may be defined by a ring shaped base 225, inner wall 210 attached to an inner diameter of the ring and outer wall 205 attached to an outer diameter of the ring, each with a sealed connection. In some example embodiments, inner wall 210 has a height H2 that is less than a height H1 of outer wall 205. Optionally a ratio H2 over H1 is defined between 0.3-1.0.

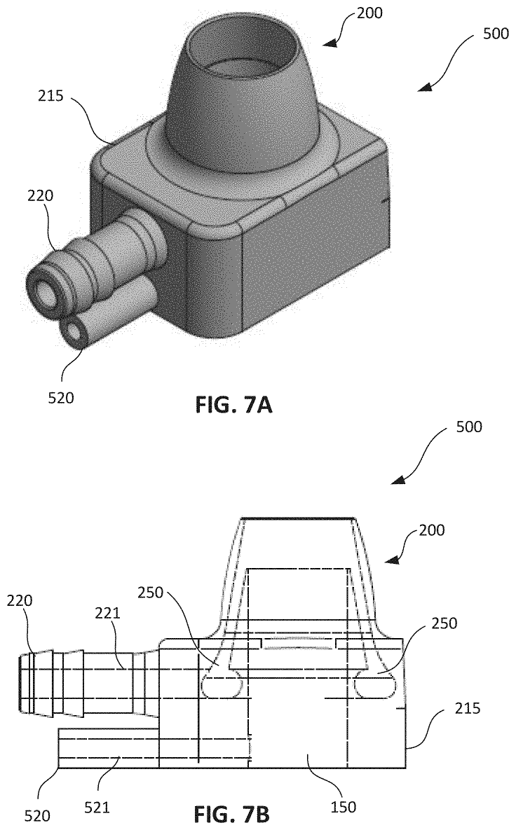

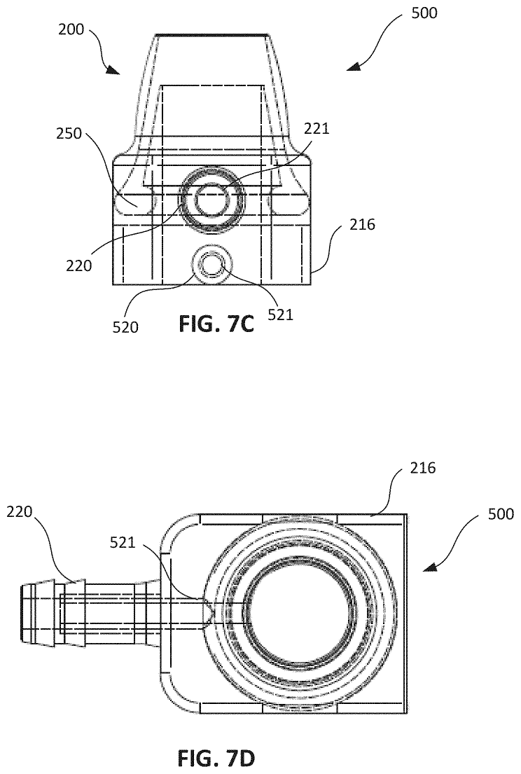

Reference is now made to FIGS. 7A, 7B, 7C and 7D showing a perspective, side, front and top view respectively of another example nasal cannula in accordance with some example embodiments of the present disclosure. According to some example embodiments, a nasal cannula 500 includes a first connector 220 defining an air flow channel 221 that is in flow communication with circumferential cavity 250 in prong 200 and may include an additional connector 520 defining an airflow channel 521 that is in fluid communication with flow through bore 150. Optionally, a base 216 provides structural support for prong 200, connector 220 and additional connector 520.

In some example embodiments, additional connector 520 may be configured to connect air flow to a sensor, e.g. pressure sensor or air sampler configured to monitor parameters while a patient is breathing. Measurements may be made while air or gas is being delivered through channel 221 into circumferential cavity 250 optionally in synchronization with a breathing rhythm and may also be made while no air or gas is being delivered. Optionally, a diameter of air flow channel 521 defined through additional connector 520 is smaller than air flow channel 221 defined through connector 220.

It is noted that although only one prong 200 is shown in nasal cannula 500 for simplicity purposes, a nasal cannula 500 may be similarly formed with a pair of prongs 200 as shown in FIGS. 1-3. Furthermore, for embodiments including a pair of prongs 200, additional connector 520 may be provided on either one or on both of prongs 200. For example, prong 200 for a left nostril may include both connectors 220 and 520 and another prong 200 for a right nostril may only include connector 220. In another example, prong 200 for a left nostril may include both connectors 220 and 520 and another prong 200 for a right nostril may also include both connectors 220 and 520. Prongs 200 for both nostrils may be formed in a single base 215 as shown for example in FIGS. 1-3.

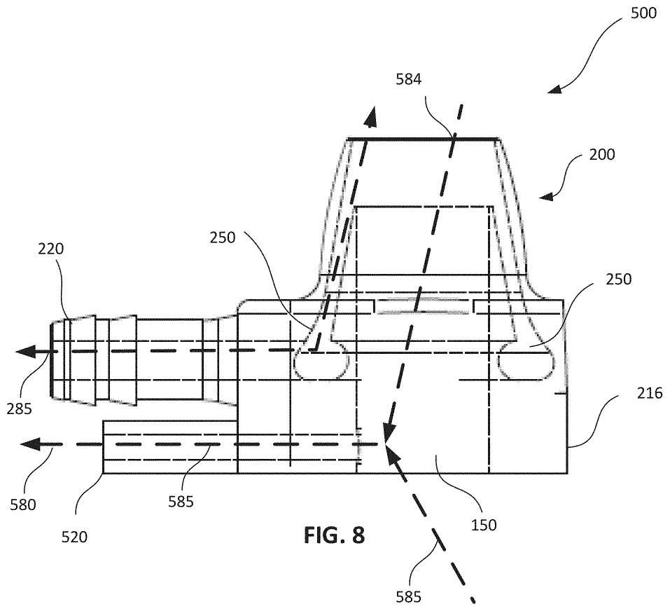

FIG. 8 is a side view of the other example nasal cannula with arrows indicating example airflow directions in accordance with some example embodiments of the present disclosure. In some example embodiments, air may be extracted from connector 520 for sensing. Optionally a vacuum may be applied to extract air from bore 150 (arrow 580). Sampled air may be air that a patient exhales (arrow 584) and may be air from the surrounding environment that a patient inhales (arrow 585). In some example embodiments, a sensor connected to connector 520 may sense parameters related to both inhalation and exhalation. In some example embodiments, sensing may be alternatively or additionally performed via connector 520.

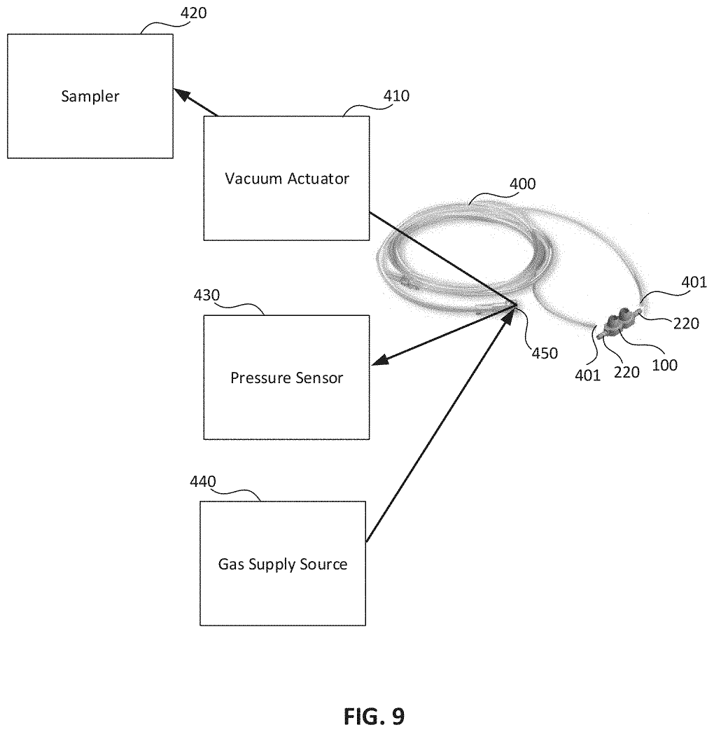

Reference is now made to FIG. 9 showing a simplified block diagram of an example nasal cannula with associated sensors in accordance with some example embodiments. According to some example embodiments, a nasal cannula 100 may be connected to tubing 400 based on attaching ends 401 to connectors 220. Tubing 400 typically includes a distal connector 450 connected to tube 400 at an end opposite an end at which nasal cannula 100 is connected. Distal connector 450 may be connected to one or more of a gas supply source 440 for delivering gas to the patient, a pressure sensor 430 configured to monitor pressure in nasal cavity during breathing, and a vacuum actuator 410, e.g. a pump for extracting air from the nasal cavity to a sampler 420 for sampling the air in the nasal cavity.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following example.

### Example

Reference is now made to the following examples, which together with the above descriptions illustrate some embodiments of the invention in a non limiting fashion.

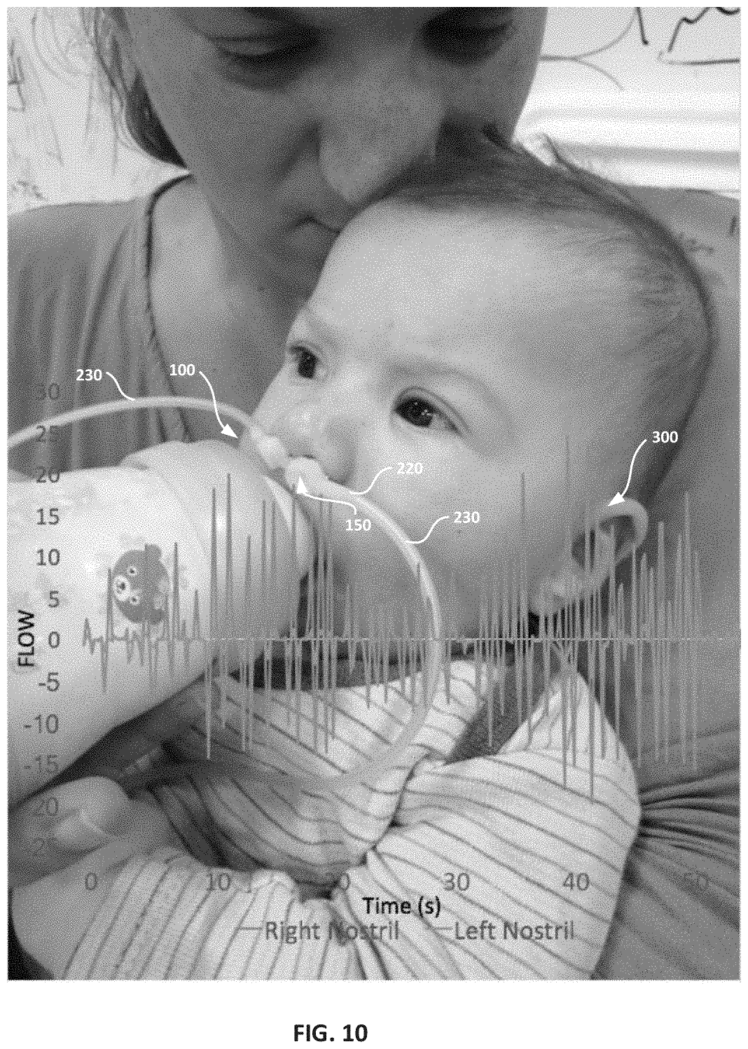

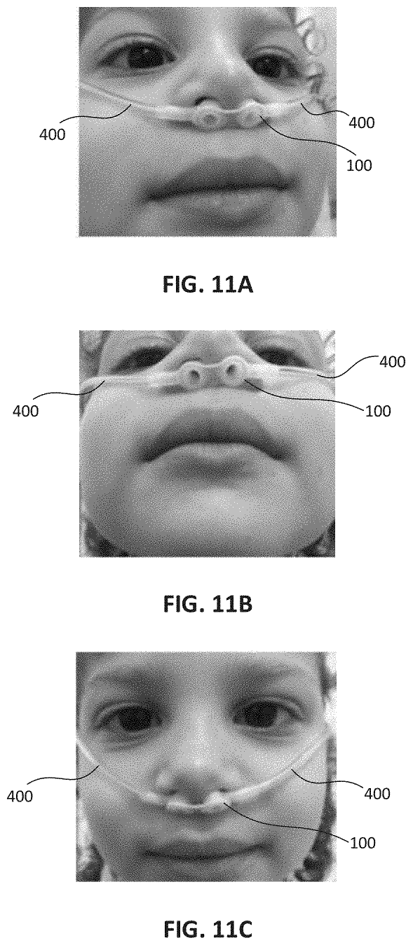

Reference is now made to FIG. 10 showing an image of an infant fitted with an example nasal cannula and to FIGS. 11A, 11B and 11C showing images of a child fitted with the example nasal cannula, both in accordance with some example embodiments of the present disclosure and a graph of example measured flow with the example nasal cannula. As can be seen in the image of FIG. 10, example nasal cannula 100 may be sized to fit an infant. While the infant may receive air or gasses delivered to nasal cannula 100 via tubes 230 and connectors 220, the infant may also freely inhale and exhale air naturally (from the surrounding environment) through bore 150 in nasal cannula 100. Graph 300 is an example graph of actual nasal airflow recorded through nasal cannula 100. The blue curve represents measurements taken from the right nostril and the orange curve represents measurements taken from the left nostril. These measurements indicate normative breathing. By providing a volume through which the infant may exhale freely, nasal cannula 100 may avoid end-distending pressure buildup and thereby the risk of gastrointestinal perforations.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention.

To the extent that section headings are used, they should not be construed as necessarily limiting. In addition, any priority document(s) of this application is/are hereby incorporated herein by reference in its/their entirety.

What is claimed is:

1. A nasal cannula comprising:
a prong formed from an inner cylindrical wall surrounded by an outer cylindrical wall and a base connecting the inner cylindrical wall to the outer cylindrical wall, wherein the inner cylindrical wall, the outer cylindrical wall and the base together define an air channel between the inner and outer cylindrical wall, wherein the inner cylindrical wall, the outer cylindrical wall and the air channel there between are configured to be received within a nostril and wherein the inner cylindrical wall defines a bore through which air may freely flow; and
a connector fluidly connected to the air channel and configured to provide flow communication between the air channel and an external device connected to the connector.

2. The nasal cannula of claim 1, wherein the inner cylindrical wall has a first height and wherein the outer cylindrical wall has a second height and wherein the first height is less than the second height.

3. The nasal cannula of claim 1, wherein the outer cylindrical wall has a truncated cone shape.

4. The nasal cannula of claim 1, wherein the inner cylindrical wall has a first diameter and wherein the outer cylindrical wall has a second diameter at its distal end with respect to the base and wherein a ratio of the first diameter over the second diameter is 0.8-1.0.

5. The nasal cannula of claim 1, wherein a width of the air channel tapers distal to the base.

6. The nasal cannula of claim 1, wherein the connector is connected to the air channel via a port formed in the outer cylindrical wall.

7. The nasal cannula of claim 1, wherein the base is configured to structurally support the prong and the connector and wherein the bore defined by the inner cylindrical wall extends through the base.

8. The nasal cannula of claim 1, wherein the inner cylindrical wall is configured to be flexible and to collapse toward the outer cylindrical wall absent flow pressure in the air channel.

9. The nasal cannula of claim 1, wherein the external device is a source of compressed gas and wherein the air channel is configured to receive gas from the external device via the connector and expel the gas through an opening between the inner cylindrical wall and the outer cylindrical wall.

10. The nasal cannula of claim 1, wherein the prong is configured to provide flow communication from the opening between the inner cylindrical wall and the outer cylindrical wall to the external device based on applying a vacuum through the connector.

11. The nasal cannula of claim 1, comprising a second connector connected to a port in the inner cylindrical wall, wherein the second connector is configured to provide flow communication between the bore and a sensor for sensing a parameter of breathing.

12. The nasal cannula of claim 1, comprising a pair of the prong, wherein the base connects the pair.

13. The nasal cannula of claim 12, comprising a pair of the connector, wherein each connector of the pair is fluidly connected to the air channel of one of the pair of the prong.

14. A method for providing respiratory assistance, the method comprising:
providing a nasal cannula, the nasal cannula comprising:
a prong formed from an inner cylindrical wall surrounded by an outer cylindrical wall, a base connecting the inner cylindrical wall to the outer cylindrical wall and an air channel formed between the inner and outer cylindrical wall, wherein the inner cylindrical wall, the outer cylindrical wall and the air channel are configured to be received within a nostril and wherein the inner cylindrical wall defines a bore through which air may freely flow;
a first connector fluidly connected to the air channel and configured to provide flow communication between the air channel and an external device connected to the connector; and
a second connector connected to a port in the inner wall, wherein the second connector is configured to provide flow communication between the bore and a sensor for sensing a parameter of breathing;
delivering air to the nasal cannula via the connector; and
sensing the parameter of breathing based on connecting the second connector to the sensing device.

15. The method of claim 14, comprising extracting air from the second connectors and sensing the parameter of breathing based on the air extracted.

16. The method of claim 15, wherein the delivering and the extracting are performed concurrently.

17. The method of claim 15, wherein the delivering and the extracting are performed consecutively.

18. The method of claim 15, wherein the extracting is synchronized to occur at a defined period in a breathing cycle.
